(19) United States
(12) Patent Application Publication
(10) Pub. No.: US 2018/0207138 A1
(43) Pub. Date: Jul. 26, 2018

(54) METHODS FOR THE TREATMENT OF TREMORS BY POSITIVE MODULATION OF SK CHANNELS

(71) Applicant: Cadent Therapeutics, Inc., Cambridge, MA (US)

(72) Inventors: Dipak Vasantrao Amrutkar, Ballerup (DK); Kelly Foster, Watertown, MA (US); Martin R. Jefson, Stonington, CT (US); Gregg F. Keaney, Lexington, MA (US); Karin Sandager Nielsen, Fredensborg (DK)

(21) Appl. No.: 15/877,918

(22) Filed: Jan. 23, 2018

Related U.S. Application Data
(60) Provisional application No. 62/449,265, filed on Jan. 23, 2017.

(51) Int. Cl. A61K 31/428; A61P 25/14

(52) U.S. Cl. CPC A61K 31/428; A61P 25/14

(57) ABSTRACT

Provided herein is the use of one or more small-conductance calcium-activated potassium channel positive modulators (SK positive modulators) for the treatment of tremors.

### RELATED APPLICATIONS

This application claims priority to U.S. Provisional Application No. 62/449,265, filed Jan. 23, 2017, the entire contents of which are incorporated herein by reference.

### BACKGROUND

Tremors are involuntary muscle contractions and relaxations involving oscillations or twitching movements of one or more body parts. Of the 20 or so tremors, essential tremor is one of the most common, affecting approximately 0.9% of the general population (Mov. Disord. 25, 534-541, 2010). Essential tremor is characterized by an action tremor of the upper limbs and, less commonly, the head, voice, and trunk (Curr. Neurol. Neurosci. Rep. 13, 353, 2013). The etiology of essential tremor is largely unknown. A family history of essential tremor can be identified in approximately half of patients (Parkinsonism Relat. Disord. 13, 333-339, 2007), suggesting a genetic component, though the underlying genetics have remained elusive. Essential tremor appears to arise from oscillatory network activity involving a loop that includes the inferior olive, the cerebellum, the thalamus, and the cortex (Clin. Neurophysiol. 123, 61-64, 2012), though it is unclear what causes this oscillatory behavior. Substantial evidence supports the idea that essential tremor is a neurodegenerative disorder. Symptoms are progressive (Arch. Neurol. 57, 1194-1198, 2000), and disease prevalence rises with increasing age (Mov. Disord. 25, 534-541, 2010). Histopathology from patients with essential tremor indicates Purkinje cell degeneration and loss and related cerebellar pathology (Mov. Disord. 31, 393-401, 2016; Arch. Neurol. 66, 1202-1208, 2009; and J. Mov. Disord. Soc. 29, 1329-1330, 2014), though there is some controversy surrounding this topic (Neurodegener. Dis. Manag. 2, 259-268, 2012).

Treatments, in the form of both medications and surgery, are available, though all of the currently available treatment options have limitations. For example, while propranolol and primidone constitute the first line medications, approximately 50% of patients fail to respond and a proportion of those that do respond do not tolerate the side effects (Neurology 86, S27.006, 2016 and Neurotherapeutics 11, 128-138, 2014). Response rates to deep brain stimulation and thalamotomy are much higher than for available medications (J. Neurol. Neurosurg. Psychiatry 86, 257-264, 2015 and Mov. Disord. 16, 464-468, 2001, though such procedures are highly invasive or involve irreversible ablation of brain tissue. Thus, there remains a large unmet medical need among patients suffering from tremors such as essential tremor.

### SUMMARY

It has now been found that small-conductance calcium-activated potassium channel positive modulators (SK positive modulators) reduce tremors in a pharmacological model of essential tremor. See e.g., FIGS. 1 and 2. Thus, provided herein is the use of SK positive modulators for treating essential tremor, and other tremors.

One embodiment of the present disclosure is a method of treating tremors in a subject using an effective amount of a SK positive modulator. The SK positive modulator may be a modulator of SK1, SK2, SK3 and/or SK4. In one aspect, the SK positive modulator is a modulator of SK2.

Also provided herein is an SK positive modulator for treating essential tremor in a subject. The SK positive modulator may be a modulator of SK1, SK2, SK3 and/or SK4. In one aspect, the SK positive modulator is a modulator of SK2.

### BRIEF DESCRIPTION OF THE FIGURES

FIG. 1 is a diagram illustrating the effect of various SK positive modulators following oral (PO) dosing on harmaline induced tremor. Panel A is chlorzoxazone (CHZ) dosed orally. Panel B is Compound 1 dosed orally. Panel C is Compound 2 dosed orally.

FIG. 2 is a diagram illustrating the % SK2 SC₁₀₀ at which SK positive modulators chlorzoxazone, Compound 1, and Compound 2 achieve efficacy in the Harmaline model.

FIG. 3 displays the efficacy and dose response of Compound 2 in percent motion power.

### DETAILED DESCRIPTION

Provided herein is the use of one or more SK positive modulators for the treatment of tremors such as e.g., essential tremor.

SK channels are members of a family of voltage-independent potassium channels that are activated by increases in intracellular Ca²⁺ via their interaction with calmodulin (Nature 395, 503-507, 1998). They are characterized by their low conductance (˜10 pS), and are a subfamily of Ca²⁺-activated K⁺ channels and the SK channel family contains 4 members—SK1, SK2, SK3, and SK4 (often referred to as intermediate conductance). These channels can be activated by Ca²⁺ entering through voltage-gated Ca²⁺ channels following an action potential, and can be important in regulating membrane excitability (Curr. Opin. Neurobiol. 15, 305-311, 2005). In cells that fire tonically, SK channels can be important in regulating pacemaking ability.

The physiological role of the SK channels has been especially studied in the nervous system, where e.g., they are key regulators of neuronal excitability and of neurotransmitter release, and in smooth muscle, where they are crucial in modulating the tone of vascular, broncho-tracheal, urethral, uterine or gastro-intestinal musculature.

A “small-conductance calcium-activated potassium channel positive modulator” or “SK positive modulator” refers to an agent which amplifies potassium channel sensitivity to calcium e.g., by increasing the current in potassium channel. These compounds include channel openers and allosteric modulators. In one aspect, a compound is determined to be an SK positive modulator by measuring the ionic current through small-conductance Ca²⁺-activated K⁺ channels using the whole-cell configuration of the patch-clamp technique in a patch-clamp set-up using HEK293 tissue culture cells expressing SK2 channels as described in Hougaard et al., British Journal of Pharmacology 151, 655-665, May 8, 2007, the entire teachings of which are incorporated herein by reference. In brief, whole cell voltage clamp recordings are established from SK2 expressing HEK293 cells and current is measured at −30 mV. Extracellular solution consists of 4 mM KCl, 144 mM NaCl, 2 mM CaCl₂, 1 mM MgCl₂, and 10 mM HEPES, adjusted to pH 7.4 by NaOH or KOH. The intracellular solution contains 154 mM KCl, 10 mM HEPES and 10 mM EGTA. Additionally, CaCl₂ is added to the intracellular solution to give calculated free concentrations of Ca²⁺ of 0.3 μM. MgCl₂ is added to give a free concentration of 1 mM Mg²⁺. In one aspect, a compound is defined to be an SK positive modulator if the compound increases current in this assay, for example, if the SC₁₀₀ value of the compound is less than or equal to 10 μM as determined by this assay. The SC₁₀₀ value is defined to be the concentration of compound that increases the basal current by 100%.

SK positive modulators include e.g., those described in U.S. Provisional Application Nos. 62/347,762 and 62/344,513, WO 2000/034244, WO 2008/123756, WO 2006/069806, WO 2008/074756, WO 2008/135591, WO 2008/135448, and U.S. Pat. No. 7,825,131,

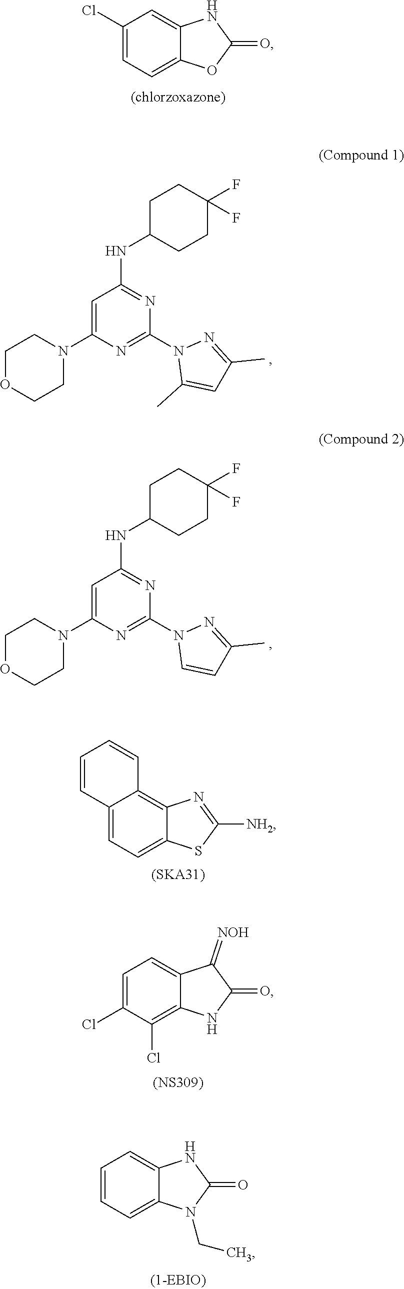

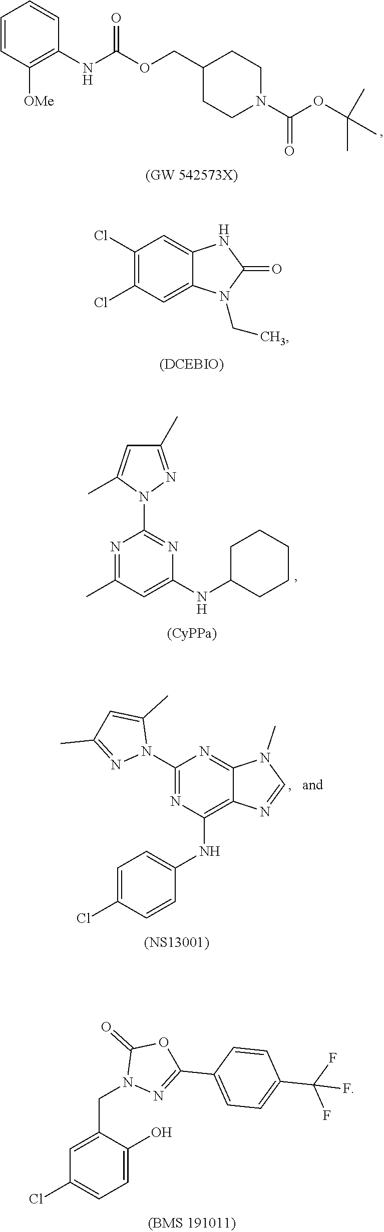

In one aspect, the SK positive modulators described herein do not contain a moiety having the formula:

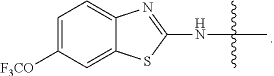 [SMILES: *Nc1nc2ccc(OC(F)(F)F)cc2s1]

Alternatively, SK positive modulators having the formula:

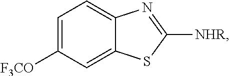

where R is hydrogen or NHR is an amide, carbamate, or urea are excluded. Specific examples can be found in e.g., WO 2016/140879 and WO 2016/140878, the contents of each of which are incorporated herein by reference. For example, compounds having the formula:

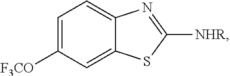

where R is defined by R¹ in WO 2016/140879 and WO 2016/140878, and where R is an amide represented by Formula XVIII in WO 2016/140879 and WO 2016/140878 are excluded.

The terms “subject” and “patient” may be used interchangeably, and means a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

The terms “treatment,” “treat,” and “treating” refer to reversing, alleviating, reducing the likelihood of developing, or inhibiting the progress of a disease or disorder, or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), i.e., prophylactic treatment. Treatment may also be continued after symptoms have resolved, for example to prevent or delay their recurrence.

The term “effective amount” or “therapeutically effective amount” includes an amount of an SK positive modulator that will elicit a biological or medical response of a subject, for example, amelioration of symptoms of essential tremor, or the slowing or delaying of progression of essential tremor. In some embodiments, the language “effective amount” includes the amount of an SK positive modulator that when administered to a subject, is effective to at least partially alleviate and/or ameliorate a tremor such as essential tremor.

Chlorzoxazone, Compound 1, and Compound 2 were evaluated in male Sprague Dawley rats for efficacy in reducing Harmaline-induced tremor. The structure of each of these compounds is duplicated below.

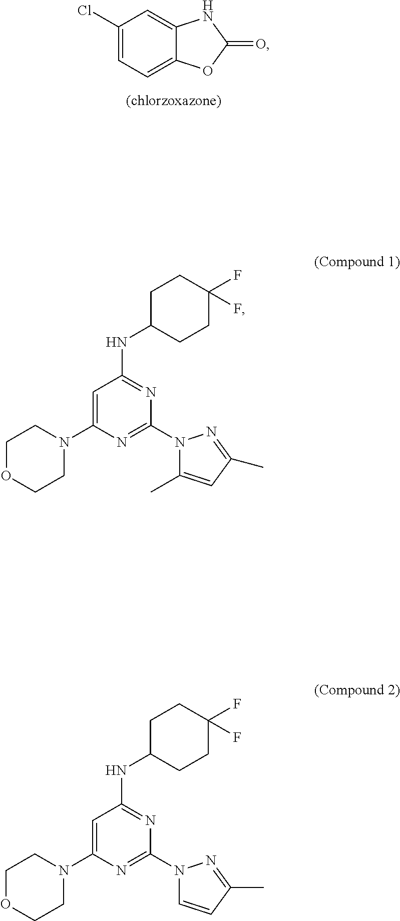 [SMILES: O=c1[nH]c2cc(Cl)ccc2o1 ; Cc1cc(C)n(-c2nc(NC3CCC(F)(F)CC3)cc(N3CCOCC3)n2)n1 ; Cc1ccn(-c2nc(NC3CCC(F)(F)CC3)cc(N3CCOCC3)n2)n1]

Animals were administered with either Vehicle or compound (orally) 30 minutes prior to Harmaline administration (t=−30 minutes). Animals were then dosed ip with 10 mg/Kg Harmaline HCl in isotonic saline (t=0 minutes). Immediately following Harmaline administration, animals were monitored for 1 hour (t=0-60 minutes) to quantify the number of tremor events. Dosages of compounds were as follows: 10 or 30 mg/Kg chlorzoxazone, 30 or 60 mg/Kg Compound 1, and 10 or 30 mg/Kg Compound 2.

Tremor events were quantified via automated capture of forelimb tremor activity and confirmed by visual observation. Prior to testing, animals were fitted with a small metal band (0.5 g) on the right forepaw and acclimated to the testing apparatus for one hour. Immediately following Harmaline administration, animals were placed in the testing apparatus and tremor events were quantified for 60 minutes. A tremor event signal was generated when the transmitter band on the animal moved within the electromagnetic field generated by a loop antenna within the testing apparatus. Outputs from the amplifier were digitized at a sampling rate of 1,000 Hz and the signal was processed and analyzed using LabView software (National Instruments). To minimize signal from ambulatory and grooming behavior, the signal was filtered with a 128-ms unweighted moving average filter, and events with amplitudes >0.5 V and lasting >300 ms in duration were counted as a tremor event. Data were analyzed in one-minute bins over the course of the test and presented as the sum of tremor events over the entire 60 minute test.

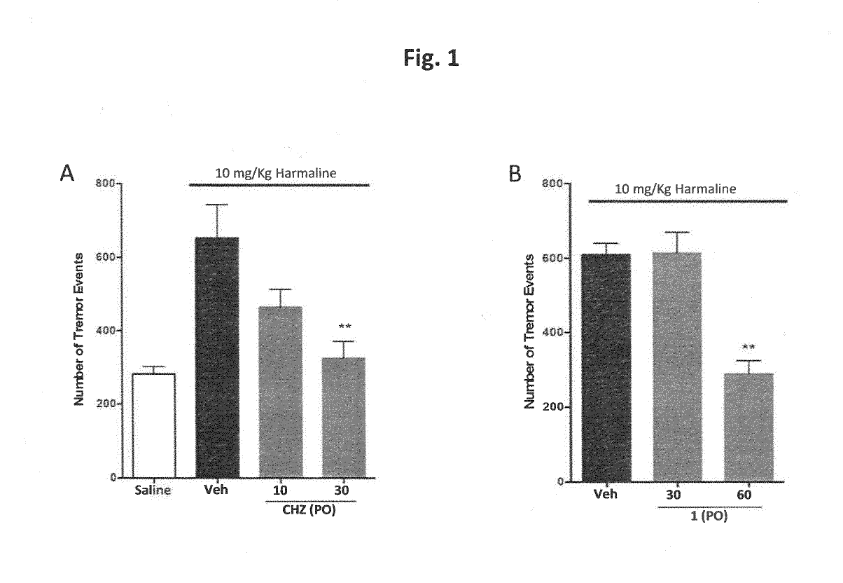

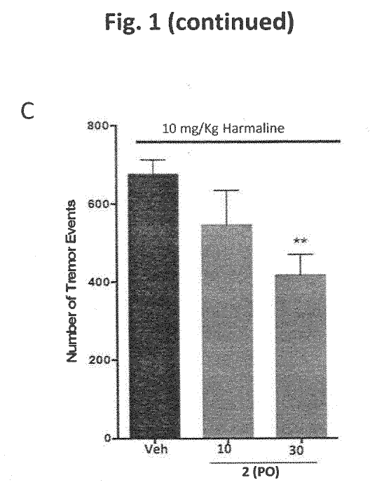

As shown by FIG. 1, Panels A-C, significant inhibition of tremors was observed at a dose of 30 mg/Kg chlorzoxazone, 60 mg/Kg Compound 1, and 30 mg/Kg Compound 2. The extent to which compounds modulate SK2 channels in vivo is expressed as % SK2 SC₁₀₀, which is the ratio of the concentration of the drug free in the brain to the measured potency of the compound against the SK2 channel. It is calculated as follows: C_{FB}=C_{MB}×BFF, where C_{MB} is the concentration of compound measured by mass spectrometry from brains harvested immediately following tremor recording (Table 1, “Measured Brain Concentration”). C_{FB} is the amount of free compound not complexed with protein and therefore free to interact with the SK2 channel (Table 1, “Calculated Free Brain Concentration”). BFF is average free fraction of compound as measured by equilibrium dialysis (Table 1, “Brain Free Fraction”). This was performed by using 1 μM of compound with 10% brain tissue homogenate in phosphate buffer saline. Incubation time was 5 hours at 37° C. and detection was by LC-MS/MS. Reference compound was carbazepine. Free drug in brain available to interact with SK2 channels (C_{FB}) is arrived at by multiplying the measured total brain level (C_{MB}) by the average free fraction (BFF).

TABLE 1

| | Minimally | Measured | Measured | Calculated | Measured | |
|---|---|---|---|---|---|---|
| | Efficacious | Brain | Brain | Free Brain | SK2 | Calculated |
| | Dose | Concentration | Free | Concentration | SC₁₀₀ | % SK2 |
| Compound | (mg/Kg) | (μM) | Fraction | (μM) | (μM) | SC₁₀₀ |
| Chlorzoxazone | 30 | 28.9 | 0.12 | 3.67 | 32.0 | 11 |
| 1 | 60 | 3.1 | 0.048 | 0.15 | 0.6 | 24 |
| 2 | 30 | 1.3 | 0.065 | 0.08 | 0.5 | 16 |

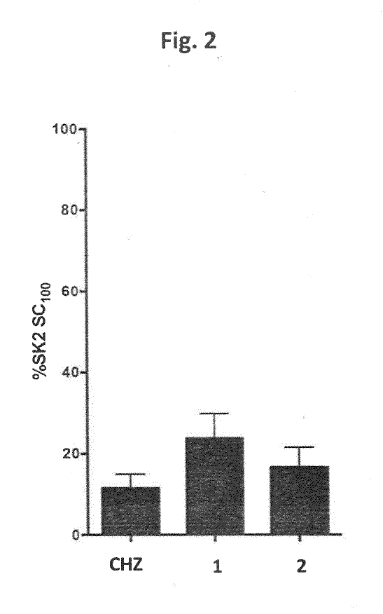

The amount of free compound is then expressed in terms of its potency against the SK2 channel as follows: % SK2 SC₁₀₀=C_{FB}/SK2 SC₁₀₀×100, where SK2 SC₁₀₀ (Table 1, “SK2 SC₁₀₀”) is the measured value of potency of the compound against SK2 channels and % SK2 SC₁₀₀ (Table 1, “% SK2 SC₁₀₀”) is the free brain concentration (C_{FB}) normalized to SK2 SC₁₀₀. Thus the % SK2 SC₁₀₀ gives a measure of the degree to which each of the compounds is modulating SK2 channels regardless of differences in potency or exposure. Importantly, all compounds displayed efficacy at a dose that represented similar modulation of the SK2 channel, regardless of potency (FIG. 2).

This above data shows that compounds from different classes, and having different potencies against SK2 channels, each modulate SK2 channels to a similar extent at efficacious doses, and also inhibit tremors (FIG. 1). This establishes that positive modulation of SK2 channels is a mechanism of action in ameliorating tremor.

Reduction of tremor with Compound 2 has also been demonstrated by measurement of whole-body tremor frequency via a force-plate accelerometer.

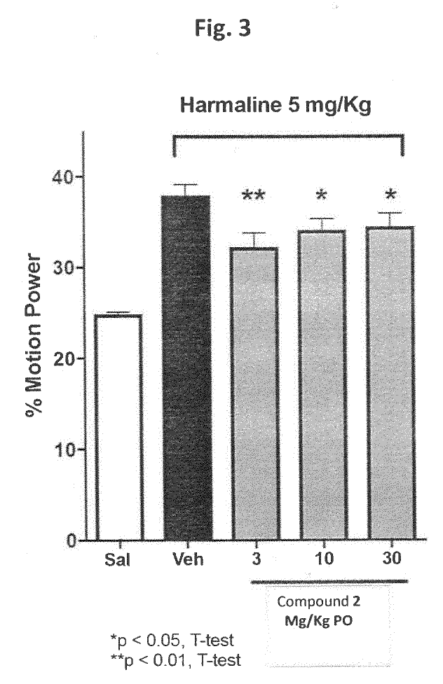

Whole body tremor was measured by a San Diego Instruments Tremor Monitor (San Diego, Calif., USA). Animals were pre-treated with 3, 10, or 30 mg/kg Compound 2 orally 30 minutes prior to intraperitoneal administration of 5 mg/kg harmaline. Tremor was measured for 30 minutes following harmaline administration, and data were analyzed by fast Fourier transform and reported as a frequency power spectrum. Harmaline induced a significant increase in the power spectrum in a band of frequencies between 10 and 14 Hz. In this range, 3, 10, and 30 mg/kg all significantly reduced tremor. Data were further analyzed by calculating the percent Motion Power (% MP), defined as the power in the 9-13 Hz band divided by the total power across the spectrum (0-30 Hz) multiplied by 100. By this analysis, 3, 10, and 30 mg/kg significantly reduced harmaline-induced tremor (harmaline+vehicle (n=13); harmaline+3 mg/kg Compound 2 (n=8), P<0.01; 10 mg/kg Compound 2 (n=16) and 30 mg/kg Compound 2 (n=13), respectively, P<0.05) (FIG. 3).

Taken together, these data show that Compound 2 significantly reduces harmaline-induced tremor measured by two different experimental designs.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating an essential tremor in a subject comprising administering to the subject an effective amount of a small-conductance calcium-activated potassium channel positive modulator (SK positive modulator), provided the SK positive modulator does not contain a moiety having the formula:

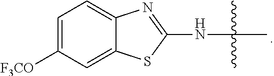 [SMILES: *Nc1nc2ccc(OC(F)(F)F)cc2s1]

2. (canceled)

3. The method of claim 1, wherein the SK positive modulator is modulator of SK2.

4. The method of claim 1, wherein the SK positive modulator is an allosteric modulator.

5. The method of claim 1, wherein the SK positive modulator is a channel opener.
